(19) United States
(12) Patent Application Publication
(10) Pub. No.: US 2001/0027293 A1
(43) Pub. Date: Oct. 4, 2001

(54) STORAGE STABLE FLUID INJECTION DEVICE AND ASSOCIATED PROCESS

(76) Inventor: ASHOK V. JOSHI, SALT LAKE CITY, UT (US)

Correspondence Address: JODY L FACTOR, FACTOR AND SHAFTAL LLC, 100 WEST MONROE STREET, SUITE 300, CHICAGO, IL, 60603

(21) Appl. No.: 09/342,851

(22) Filed: Jun. 29, 1999

(51) Int. Cl. A61M 37/00

(52) U.S. Cl. 604/131

(57) ABSTRACT

A storage stable needleless fluid injection device comprising a reaction chamber capable of retaining a predetermined quantity of a chemical reagent that generates pressure when the chemical reagent is at least one of reacted, ignited, exploded, and decomposed, an initiator for initiating reaction, ignition, explosion and/or decomposition of the chemical reagent, a fluid retention chamber capable of retaining a fluid, wherein the fluid retention chamber includes an aperture, and means for dispensing a fluid retained within the fluid retention chamber out of the aperture, and in turn, into a human or animal body, or other object. The device is capable of generating pressures in excess of 500 p.s.i. with the use of only very small amounts of the chemical reagent. Such high pressures enable needless injections to deep sub-dermal locations within a body or other desired environments.

### BACKGROUND OF THE INVENTION

1. Field of the Invention

The present invention relates in general to fluid injection devices, and more particularly, to needleless injection devices that are driven by, for example, pressure generated from an explosive reaction and/or ignition of a chemical composition.

2. Background Art

Fluid injection devices have been known in the art for several years. Furthermore, needleless fluid injection devices, such as medicinal syringes, driven by air, carbon dioxide, or spring mechanisms are likewise well known. Such devices may be used, for example, for delivering vaccinations or medication to a human and/or an animal. While utilization of these fluid injection devices has become increasingly popular among the medical and veterinarian disciplines, problems have been identified with respect to, among other things, their performance limitations. Notably, fluid injection devices that are driven by cartridges filled with air or carbon dioxide, or alternatively by spring mechanisms are typically only capable of operating at or below approximately 100 p.s.i., which can be problematic inasmuch as certain medical and/or therapeutic applications require substantially more pressure to inject the fluid to a desired deep level. Moreover, the present devices with CO₂ or air cartridges leak and lose the ability to reach even 100 p.s.i. The shelf life is poor. Accordingly, it is desirable to have a storage stable, high pressure, needleless syringe. To the best of applicant's knowledge such storage stable, high pressure deep injections are only presently attainable by invasive, needled syringes.

### SUMMARY OF THE INVENTION

The present invention is directed to a fluid injection device comprising a) a reaction chamber capable of retaining a predetermined quantity of a chemical reagent that generates pressure when the chemical reagent is reacted, ignited, exploded, or decomposed; b) an initiator for initiating at least a reaction, ignition, explosion, or decomposition of the chemical regent; c) a fluid retention chamber capable of retaining a fluid, wherein the fluid retention chamber includes an aperture; and d) means for dispensing a fluid retained within the fluid retention chamber out of the aperture, and in turn, to inject the fluid.

In a preferred embodiment of the invention, the reaction chamber includes a chemical reagent selected from the group consisting essentially of azides, oxides, superoxides, peroxides, perchlorates, hydroxides, hydrides, nitrates, nitrides, metal powders, explosive compositions and mixtures thereof.

In another preferred embodiment of the invention, the chemical reagent consists essentially of an azide mixed with a metal oxide. In this embodiment the azide preferably consists of sodium azide.

In yet another preferred embodiment of the invention, the chemical reagent consists of explosive compositions.

Preferably the initiator is selected from the group consisting of an electrical or mechanical spark ignitor, an electrical resistor ignitor, a mechanical compression ignitor, or combinations thereof.

In another preferred embodiment of the invention, the fluid is injected into a body of, for example, a human and/or an animal.

In a preferred embodiment of the invention, the means for dispensing the fluid retained within the fluid retention chamber comprises a fluid dispensing one-way valve.

In another preferred embodiment of the invention, the means for dispensing a fluid retained within the fluid retention chamber comprises a movable member associated with the fluid retention chamber. In this embodiment the movable member can comprise, for example, an elastomeric/expandable membrane or a plunger.

The fluid injection device can also be configured with a needle that is associated with the aperture of the fluid retention chamber.

Preferably the fluid injection device further comprises a pressure relief valve as well as a clamp for stabilizing the device during operation.

The present invention is also directed to a process for injecting a fluid comprising the steps of: a) initiating at least one of reaction, ignition, explosion, and decomposition of a predetermined quantity of a chemical reagent retained within a reaction chamber; b) generating pressure from at least one of the reacted, ignited, exploded or decomposed chemical reagent; c) displacing a member associated with a fluid retention chamber with the generated pressure, to in turn, dispense a predetermined amount of fluid out of an aperture of the fluid retention chamber; and d) injecting the fluid.

In a preferred embodiment of the invention, the step of initiating reaction of the chemical reagent includes the step of igniting a portion of the chemical reagent with heat generated from an electrical resistor.

In another preferred embodiment of the invention, the step of initiating reaction of the chemical reagent includes the step of igniting a portion of the reagent with a spark from a mechanical or electrical sparker.

In yet another preferred embodiment of the invention, the step of initiating reaction of the chemical reagent includes the step of igniting a potion of the reagent at predetermined intervals.

Preferably the step of generating pressure includes the step of generating a gaseous species from at least one of the group consisting essentially of azides, oxides, superoxides, peroxides, perchlorates, hydroxides, hydrides, nitrates, nitrides, metal powders, explosive compositions and mixtures thereof. In this embodiment the step of generating pressure can include the step of generating nitrogen from decomposing an azide. In this embodiment the step of generating pressure can also include the step of generating at least one of water vapor, nitrogen, and carbon dioxide from the group consisting essentially of carbonates, hydroxides, hydrides, nitrides, nitrates, metal powders, and mixtures thereof.

In another preferred embodiment of the invention, the step of displacing the member associated with the fluid retention chamber includes the step of displacing, for example, an elastomeric/expandable membrane or a plunger.

### BRIEF DESCRIPTION OF THE DRAWINGS

The invention will now be described with reference to the drawings wherein:

FIG. 1 is a schematic representation of a first embodiment of a fluid injection device in accordance with the present invention;

FIG. 2 is a schematic representation of a second embodiment of a fluid injection device in accordance with the present invention;

FIG. 3 is a schematic representation of a third embodiment of a fluid injection device in accordance with the present invention;

FIG. 4 is a schematic representation of a fourth embodiment of a fluid injection device in accordance with the present invention; and

FIG. 5 is a schematic representation of a fifth embodiment of a fluid injection device in accordance with the present invention.

### DETAILED DESCRIPTION OF THE INVENTION

While this invention is susceptible of embodiment in many different forms, there is shown in the drawings and will herein be described in detail several specific embodiments with the understanding that the present disclosure is to be considered as an exemplification of the principles of the invention and is not intended to limit the invention to the embodiments illustrated. Moreover, it will be understood that like or analogous elements are identified throughout the specification and drawings by like reference characters.

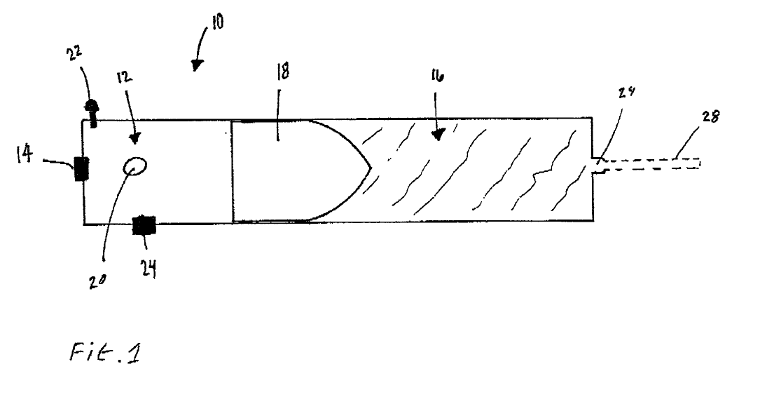

Referring now to the drawings and to FIG. 1 in particular, fluid injection device 10 generally comprises reaction chamber 12, initiator 14, fluid retention chamber 16, and displacable member or plunger 18. It will be understood that fluid injection device 10 can be configured as a disposable, single-use unit, or, alternatively, as a reusable, multi-use unit.

Reaction chamber 12 preferably retains chemical reagent 20 which, as will be discussed in detail below, can comprise any one of a number of chemical reagents or compositions. Chemical reagent 20 is preferably formed into a pellet or pill configuration. Of course, other configurations that would be known to those having ordinary skill in the art are likewise contemplated for use, including, for example, formed and unformed powders, gels, and liquids. Reaction chamber 12 may be fabricated from any one of a number of materials, including glass, metal, plastic and other synthetic resins. The only limitation with regard to fabrication materials of reaction chamber 12 is that the material must be sufficiently thermally and mechanically stable so as to not melt, fracture, or otherwise degrade during a rapid exothermic reaction—as can occur during the reaction, ignition, explosion and/or decomposition of chemical reagent 20.

Reaction chamber 12 may also include a conventional pressure relief valve 22 to relief any pressure build up during, for example, an inadvertent pressure generation sequence. Such a valve may also be used to charge reaction chamber 12 with, for example, a gas to assist in the preservation and chemical integrity of chemical reagent 20 during storage or environments of extreme temperature and/or humidity. It will be understood that if reaction chamber 12 is pressurized through pressure relief valve 22, displacable member 18 can be configured with a locking mechanism (not shown) to preclude fluid discharge while chamber 12 is under positive pressure.

Initiator 14 is preferably positioned within chamber 12 and serves to initiate reaction, ignition, explosion, and/or decomposition of chemical reagent 20. Initiator 14 can comprise, for example, an electrical or mechanical spark ignitor, an electrical resistor ignitor, or a mechanical compression ignitor. When an electrical ignitor is used, a small commercial battery, a solar cell, a direct current source and/or an alternating current source can be associated with device 10 to generate energy for the ignitor. In the case of mechanical ignitors, a user will typically depress a button or manipulate a switch and/or latch to provide the appropriate energy. As will be discussed in greater detail below, initiator 14 will serve to initiate at least one of reaction, ignition, explosion and/or decomposition of at least a portion of chemical reagent 20 by, for example, heat or a “spark.” To be sure, while initiators that generate heat or a “spark” have been disclosed, for illustrative purposes only, any one of a number of initiating source are likewise contemplated for use so long as the initiator provides enough energy to facilitate reaction of chemical reagent 20. While initiator 14 has been disclosed as being directly associated with reaction chamber 12 it is further contemplated that initiator 14 can be positioned away from chamber 12 and connected to chamber 12 through conductive means, such as wire.

Fluid retention chamber 16 is preferably isolated from reaction chamber 12 by displacable member 18. Fluid retention chamber 16 includes at least one aperture 24 for discharging a fluid retained within chamber 16, such as vaccinations and medicinal products. Fluid retention chamber 16 can be fabricated from any one of a number of materials, including glass, metal, plastic and other synthetic resins. Fluid retention chamber 16 can be optionally fitted with, among other things, needle 28. However, preferably the fluid injection device will dispense fluid to desired levels without the assistance of a needle. While a “fluid” has been disclosed, for illustrative purposes only, as the medium which is dispensed from chamber 16, it will be understood that gels, powders, and solids may likewise be dispensed.

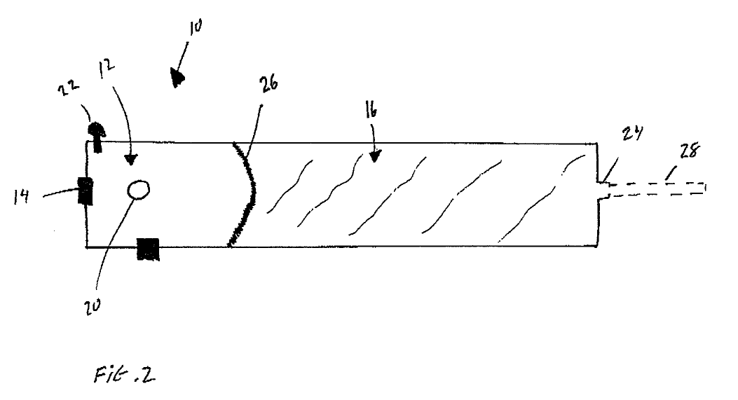

Displacable member 18 is preferably disposed between chambers 12 and 16 and is operatively displaced by pressure generated within chamber 12 as a result of reaction, ignition, explosion and/or decomposition of chemical reagent 20. Upon such displacement, the fluid retained within chamber 16 is forced out of aperture 24. While displacable member 18 has been disclosed as a plunger, for illustrative purposes only, as the mechanism by which fluid may be dispensed from chamber 16, other injection mechanisms are likewise contemplated for use. For example, as shown in FIG. 2, displacable member 18 can be exchanged with an elastomer or expanding membrane 26. Alternatively, as shown in FIG. 3, displacable member 18 can also be exchanged with one-way pressure relief valve 27.

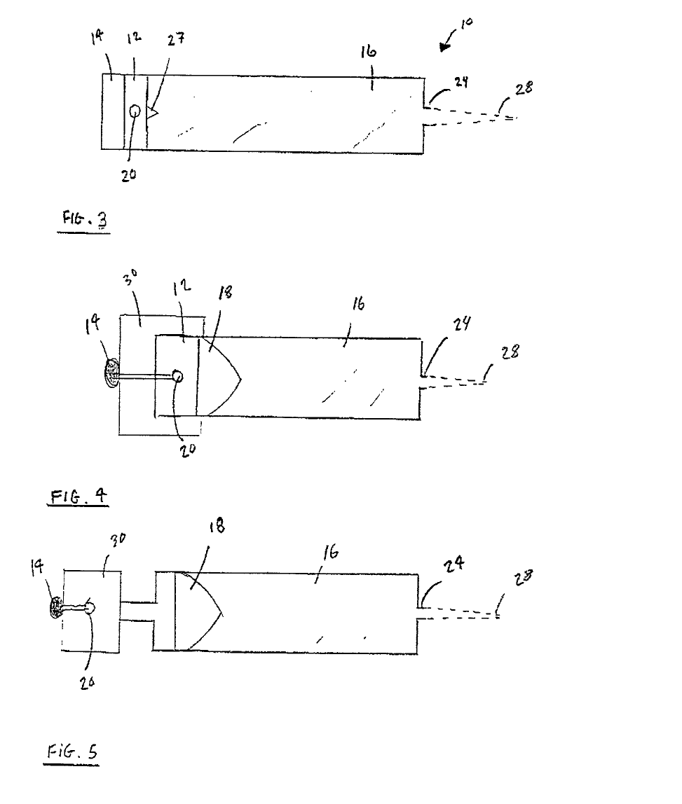

As shown in FIG. 4, fluid injection device 10 can also be configured with heat sink 30 which at least partially surrounds reaction chamber 20. The heat sink serves to, among other things, absorb and dissipate heat generated from reacting, igniting, exploding, and/or decomposing the chemical reagent or explosive mixture. Alternatively, as shown in FIG. 5, fluid injection device 10 can also be configured so that heat sink 30 also comprises the reaction chamber itself, as opposed to working in combination with a separate, albeit associated reaction chamber, such as shown in FIG. 4.

As previously discussed, chemical reagent 20 is preferably retained within to reaction chamber 12. Chemical reagent 20 is shown in FIGS. 1 and 2, for illustrative purposes only, as comprising an azide species. Azides are preferred because, upon decomposition, they generate a large amount of gas from a relatively small amount of reagent. In fact, gas generation is so substantial that pressure levels can rapidly exceed 1,000 p.s.i., which is an increase in over 900% relative to present, commercially available needleless syringes. While not shown, when pressure levels in the range of 1,000 p.s.i. are achieved, a clamp can be associated with injection device 10 to stabilize the device at such elevated conditions.

Examples of some suitable azide species include alkali metal azides, such as LiN₃, NaN₃, KN₃, RbN₃, CsN₃, and FrN₃, as well as alkaline earth metal azides. While specific, preferred azide species have been disclosed, it will be understood that other azide species (as well as non-azide species) known to those having ordinary skill in the art are likewise contemplated for use—provided such species are capable of generating pressures sufficient to deliver fluids to, for example, sub-dermal levels.

To enhance the decomposition of the azide species, oxygen, usually in the form of a metal oxide, is preferably present to participate in the decomposition. For example sodium azide and cupric oxide (copper II oxide) readily react to generate nitrogen gas according to the following chemical reaction:

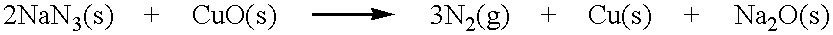

Alternatively, sodium azide and ferric oxide (iron III oxide) can react to readily generate nitrogen gas according to the following chemical reaction:

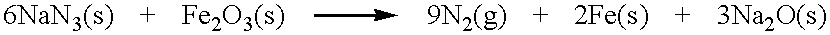

As can be seen from the above identified reactions, one mole of sodium azide generates 1.5 moles of nitrogen gas. As such, only a very small amount of azide species is needed to deliver a fluid retained within chamber 16 of device 10. Moreover, inasmuch as only a small amount of azide species is required to generate a substantial amount of nitrogen, and in turn, deliver a fluid, very small devices can be constructed. In addition, because such a large quantity of gas is generated in a kinetically fast reaction, the rate at which fluid can be dispensed or delivered is extremely high, and the level to which the fluid reaches is significantly deeper (deep sub-dermal) than conventional needleless devices.

To illustrate how very little chemical reagent is needed to generate a substantial quantity of gas, the following reaction table is provided:

| Compound | moles | MW | Vol(ml) | Mass (g) |
|---|---|---|---|---|
| 2NaN₃(s) | 0.00030 | 65.00999 | — | 0.0195 |
| CuO(s) | 0.00015 | 79.54540 | — | 0.0119 |
| 3N₂(g) | 0.00045 | 42.02022 | 10.00000 | — |
| Cu(s) | 0.00015 | 63.54600 | — | 0.0095 |
| Na₂O(s) | 0.00015 | 61.97894 | — | 0.0093 |

As can be seen from the table above, less than 0.04 grams of reagents (0.0195 NaN₃+0.0119 CuO) is needed to generate 10 mls of nitrogen gas. As such, the reagents can be formed into extremely small pellets. Of course, the amount of reagents, and in turn, the size of the reagent pellet can be varied, depending upon the amount of fluid being injected and the depth to which such fluid is being injected. It will be understood that using the ideal gas law and conventional chemical stoichiometry, one having ordinary skill in the art will be able to generate the necessary amount of gas—depending upon the particular application.

It will be understood that azide species are by no means the only acceptable reagents for generating pressure as a result of reaction, ignition, explosion, and/or decomposition of the chemical reagent. For example, oxides, peroxides, superoxides, and perchlorates, hydroxides, hydrides, nitrates, nitrides, metal powders, organic and inorganic explosive compositions and mixtures thereof are likewise contemplated for use, including compositions disclosed in: U.S. Pat. No. 3,741,585; U.S. Pat. No. 3,837,942; U.S. Pat. No. 4,021,275; U.S. Pat. No. 4,096,003; U.S. Pat. No. 4,300,962; U.S. Pat. No. 4,339,288; U.S. Pat. No. 4,401,490; U.S. Pat. No. 4,456,494; U.S. Pat. No. 4,507,161; U.S. Pat. No. 4,764,230; U.S. Pat. No. 5,074,939; U.S. Pat. No. 5,472,531; U.S. Pat. No. 5,529,649; and U.S. Pat. No. 5,587,553—all of which are herein incorporated by reference.

The present invention is also directed to a process for injecting fluid retained within fluid injecting device 10. In particular, the process begins by initiating reaction, ignition, explosion, and/or decomposition of a predetermined quantity of a chemical reagent, such as an azide species. The amount of chemical reagent that is used will depend, at least partially, upon how much gas is being generated and will vary depending upon the application. Initiating reaction can occur by any one of a number of mechanisms. However, several preferred mechanisms include mechanical or electrical sparking, heat generated from an electrical resistor, or mechanical compression. Such initiators are well known in the art and are commercially available from numerous sources.

The next step of the process is generating pressure, preferably nitrogen or oxygen, from the reacting, igniting, exploding, and/or decomposing of the chemical reagent. The generated pressure is caused by the rapid reaction occurring within reaction chamber 12. As previously discussed, the pressure which is generated will depend, at least partially, upon the combination of the chemical reagents or explosive mixtures being used—i.e. azides generate nitrogen, peroxides generate oxygen, and combinations of carbonates and hydroxides generate carbon dioxide and water vapor.

Once the pressure has been generated, this pressure will be exerted upon displacable member 18, or alternatively, elastomeric or expandable membrane 26, thus displacing it away from reaction chamber 12. Such displacement, in turn, dispenses a predetermined amount of fluid out of aperture 24, and in turn, injects the fluid, for example, into a body of a human, an animal, or alternatively, a desired environment. The term “predetermined” has been used because while not quantified, any amount of fluid can be dispensed depending upon the application. While not shown, fluid retention chamber 16 can be graduated so as to provide a user with the ability to charge chamber 16 with a precise amount of fluid.

If desirous, the above disclosed process can be repeated multiple times within one or more injection periods. For example, reaction chamber 12 can be charged with multiple units of chemical reagent 20, the reaction, ignition, explosion, and/or decomposition of which can be selectively initiated at random or at predetermined time intervals—depending upon the specific application.

The foregoing description and drawings merely explain and illustrate the invention and the invention is not limited thereto except insofar as the appended claims are so limited, as those skilled in the art who have the disclosure before them will be able to make modifications and variations therein without departing from the scope of the invention.

What is claimed is:

1. A storage stable fluid injection device comprising:
a reaction chamber capable of retaining a predetermined quantity of a chemical reagent that generates pressure when the chemical reagent is at least one of reacted, ignited, exploded, and decomposed;
an initiator for initiating at least one of reaction, ignition, explosion, and decomposition of the chemical regent;
a fluid retention chamber capable of retaining a fluid, wherein the fluid retention chamber includes an aperture; and
means for dispensing a fluid retained within the fluid retention chamber out of the aperture, and in turn, injecting the fluid.

2. The fluid injection device according to claim 1, wherein the reaction chamber includes a chemical reagent selected from the group consisting essentially of azides, oxides, superoxides, peroxides, perchlorates, hydroxides, hydrides, nitrates, nitrides, carbonates, metal powders, explosive compositions and mixtures thereof.

3. The fluid injection device according to claim 2, wherein the chemical reagent consists essentially of an azide mixed with a metal oxide.

4. The fluid injection device according to claim 3, wherein the chemical reagent consists of sodium azide.

5. The fluid injection device according to claim 2, wherein the chemical reagent is selected from the group consisting of explosive compositions.

6. The fluid injection device according to claim 1, wherein the initiator is selected from the group consisting of an electrical or mechanical spark ignitor, an electrical resistor ignitor, a mechanical compression ignitor, and combinations thereof.

7. The fluid injection device according to claim 1, wherein the fluid is injected into a body.

8. The fluid injection device according to claim 1, wherein the means for dispensing the fluid retained within the fluid retention chamber comprises a fluid dispensing one-way valve.

9. The fluid injection device according to claim 1, wherein the means for dispensing a fluid retained within the fluid retention chamber comprises a movable member associated with the fluid retention chamber.

10. The fluid injection device according to claim 9, wherein the movable member comprises an elastomeric membrane.

11. The fluid injection device according to claim 9, wherein the movable member comprises a plunger.

12. The fluid injection device according to claim 1, further comprising a needle associated with the aperture of the fluid retention chamber.

13. The fluid injection device according to claim 1, further comprising a pressure relief valve.

14. The fluid injection device according to claim 1, further including a clamp for stabilizing the device during operation.

15. A process for injecting a fluid comprising the steps of:
initiating at least one of reaction, ignition, explosion, and decomposition of a predetermined quantity of a chemical reagent retained within a reaction chamber;
generating pressure from at least one of the reacted, ignited, exploded and decomposed chemical reagent;
displacing a member associated with a fluid retention chamber with the generated pressure, to in turn, dispense a predetermined amount of fluid out of an aperture of the fluid retention chamber; and
injecting the fluid.

16. The process according to claim 15, wherein the step of initiating includes the step of igniting a portion of the chemical reagent with heat generated from an electrical resistor.

17. The process according to claim 15, wherein the step of initiating includes the step of igniting a portion of the reagent with a spark from a mechanical or electrical sparker.

18. The process according to claim 15, wherein the step of initiating includes the step of igniting a potion of the reagent at predetermined intervals.

19. The process according to claim 15, wherein the step of generating pressure includes the step of generating a gaseous species from at least one of the group consisting essentially of azides, oxides, superoxides, peroxides, perchlorates, hydroxides, hydrides, nitrates, nitrides, metal powders, explosive compositions and mixtures thereof.

20. The process according to claim 15, wherein the step of generating pressure includes the step of generating nitrogen from decomposing an azide.

21. The process according to claim 15, wherein the step of generating pressure includes the step of generating at least one of water vapor, nitrogen, and carbon dioxide from the group consisting essentially of carbonates, hydroxides, hydrides, nitrides, nitrates, metal powders, and mixtures thereof.

22. The process according to claim 15, wherein the step of displacing the member associated with the fluid retention chamber includes the step of displacing an elastomeric membrane.

23. The process according to claim 15, wherein the step of displacing the member associated with the fluid retention chamber includes the step of displacing a plunger.
